(19) United States
(12) Patent Application Publication
(10) Pub. No.: US 2016/0256057 A1
(43) Pub. Date: Sep. 8, 2016

(54) PULSE MONITORING

(71) Applicants: Brian Michael Gordon, Niskayuna, NY (US); Howard Schlossberg, Niskayuna, NY (US); Herbert Reich, Niskayuna, NY (US)

(72) Inventors: Brian Michael Gordon, Niskayuna, NY (US); Howard Schlossberg, Niskayuna, NY (US); Herbert Reich, Niskayuna, NY (US)

(21) Appl. No.: 14/716,006

(22) Filed: May 19, 2015

Related U.S. Application Data
(60) Provisional application No. 62/129,695, filed on Mar. 6, 2015.

(51) Int. Cl. A61B 5/024; A61B 5/00

(52) U.S. Cl. CPC A61B 5/024; A61B 5/0004; A61B 5/746

(57) ABSTRACT

The present invention relates generally to pulse monitoring and, more particularly, to a device, system, method, and program product for monitoring a user's pulse. In one embodiment, the invention provides a pulse monitoring device comprising: a device for detecting a pulse of an individual; and a transmission device for transmitting data related to the detected pulse.

### CROSS-REFERENCE TO RELATED APPLICATIONS

This application claims the benefit of co-pending U.S. provisional patent application Ser. No. 62/129,695, filed 6 Mar. 2015, which is hereby incorporated herein as though fully set forth.

### TECHNICAL FIELD

The present invention relates generally to pulse monitoring and, more particularly, to a device, system, method, and program product for monitoring a user's pulse, transmitting data related to the pulse to a communication device, and, in the case that the user's heart rate and/or heart beat is determined to be outside a predetermined parameter, alert the user and/or initiate a communication using the communication device.

### SUMMARY

In one embodiment, the invention provides a pulse monitoring system comprising: a monitoring device for monitoring a pulse of an individual, the device being operable to detect a pulse and transmit data related to the detected pulse; a communication device operable to receive data from the monitoring device and initiate a communication; and a program product which, when executed, is operable to carry out a method comprising: receiving data related to the detected pulse; analyzing the data related to the detected pulse and determining either or both of a heart rate or a heart beat regularity; determining whether the data are indicative of a heart rate or heart beat outside a predetermined parameter; and in the case that the data are indicative of a heart rate or heart beat being outside a predetermined parameter, initiating at least one of the following events: an alert to the individual, a communication, a transmission of data, or a storing of data.

In another embodiment, the invention provides a method of monitoring a pulse of an individual, the method comprising: receiving data related to a detected pulse of an individual; analyzing the data related to the detected pulse and determining either or both of a heart rate or a heart beat regularity; determining whether the data are indicative of a heart rate or heart beat being outside a predetermined parameter; and in the case that the data is indicative of a heart rate or heart beat outside a predetermined parameter, initiating at least one of the following events: an alert to the individual, a communication, a transmission of data, or a storing of data.

In still another embodiment, the invention provides a pulse monitoring device comprising: a device for detecting a pulse of an individual; and a transmission device for transmitting data related to the detected pulse.

In still yet another embodiment, the invention provides a program product stored on a non-transitory computer-readable storage medium, which when executed, is operable to carry out a method, the method comprising: receiving data related to a detected pulse of an individual; analyzing data related to a detected pulse; determining whether the data are indicative of a heart rate or a heart beat outside a predetermined parameter; and initiating at least one of the following events: an alert to an individual, a communication, a transmission of data, or a storing of data.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIGS. 1 and 2 show perspective views of a pulse monitoring device according to one embodiment of the invention;

FIG. 3 shows a schematic view of a pulse monitoring system according to an embodiment of the invention;

FIG. 4 shows a flow diagram of a method according to an embodiment of the invention;

FIG. 5 shows a communication device used in accordance with an aspect of the method of FIG. 4;

FIG. 6 shows a detailed view of a portion of the flow diagram of FIG. 4;

FIG. 7 shows a communication device used in accordance with another aspect of the method of FIG. 4;

FIG. 8 shows a flow diagram of a method according to another embodiment of the invention;

FIG. 9 shows a flow diagram of a method according to yet another embodiment of the invention;

FIG. 10 shows a communication device used in accordance with an aspect of the method of FIG. 9;

FIG. 11 shows a flow diagram of a method according to still yet another embodiment of the invention;

FIG. 12 shows a communication device used in accordance with an aspect of the method of FIG. 11;

FIG. 13 shows a flow diagram of a method according to another embodiment of the invention;

FIG. 14 shows a communication device used in accordance with an aspect of the method of FIG. 13;

FIG. 15 shows a flow diagram of a method according to yet another embodiment of the invention; and

FIG. 16 shows a schematic view of a system according to an embodiment of the invention.

It is noted that the drawings of the invention are not to scale. The drawings are intended to depict only typical aspects of the invention, and therefore should not be considered as limiting the scope of the invention. In the drawings, like numbering typically represents like elements between and among the drawings.

### DETAILED DESCRIPTION

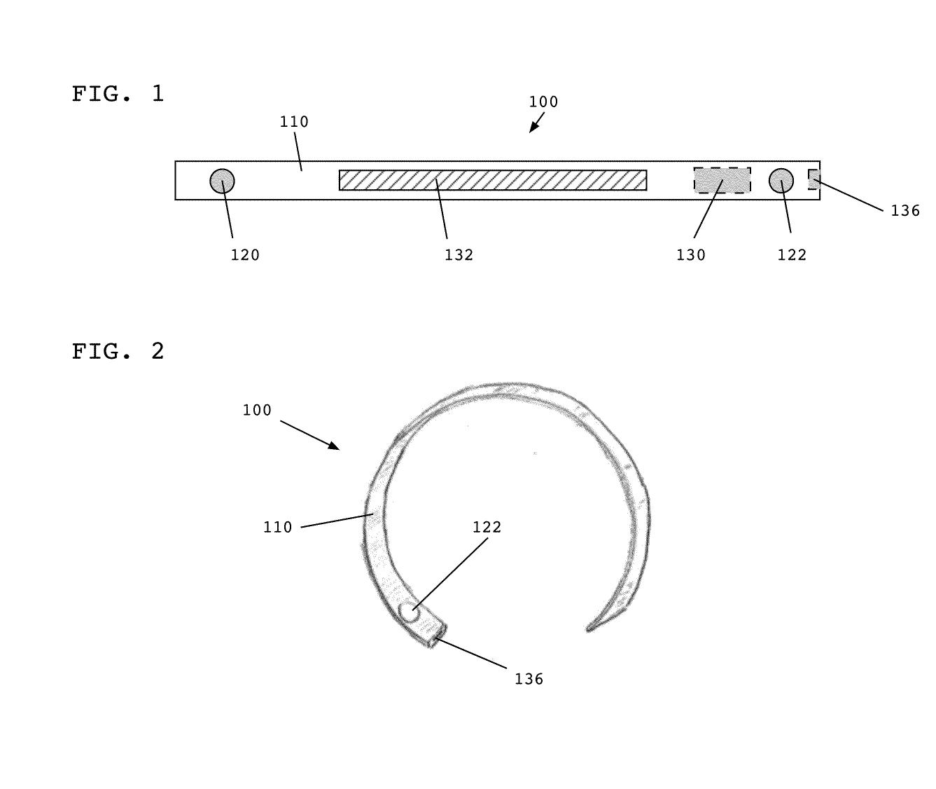

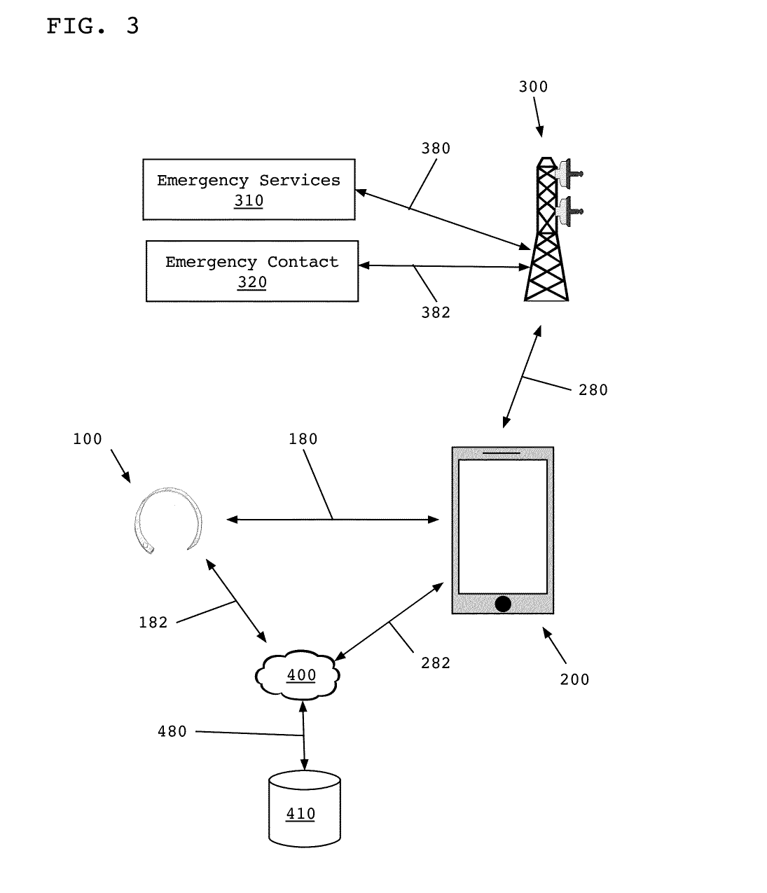

Turning now to the drawings, FIG. 1 shows a perspective view of a device 100 according to an embodiment of the invention. Device 100 includes an elongate body 110 at either end of which is disposed a pulse sensor 120,122, as may be known in the art. As will be described in greater detail below, elongate body 110 is, in some embodiments of the invention, deformable such that it may be formed into a substantially circular or ovoid shape along its length, as shown, for example, in FIG. 2. One skilled in the art will recognize that, once so formed, pulse sensors 120,122 may be placed in contact with a wearer's wrist, thereby enabling sensing of the wearer's pulse. Although a pair of pulse sensors 120,122 are shown in FIGS. 1 and 2, and are shown disposed adjacent ends of elongate body 110, this is neither necessary nor essential. Any number of pulse sensors may be employed and may be employed at any number of locations along elongate body 110. In some embodiments of the invention, for example, device 100 may include a circular or ovoid body without ends and a plurality of pulse sensors disposed along its interior. Other variations and modifications will be apparent to one skilled in the art in view of this disclosure and are within the scope of the invention.

Returning to FIG. 1, device 100 may include a number of additional features. For example, body 110 may include a Bluetooth device 130 or similar device for pairing device 100 with an electronic device, such as a mobile telephone. In some embodiments of the invention, body 110 may include an alert mechanism 132, such as, for example, a mechanism capable of providing a vibratory signal, a visual signal, and/or an auditory signal to a wearer or to those nearby the wearer. Some embodiments of the invention may include a USB or similar port 136 for charging a battery providing electrical power to device 100 and/or transferring data to or from device 100. In other embodiments of the invention, device 100 may include a chamber (not shown) for housing a removable battery device.

In still other embodiments of the invention, device 100 may include a display device (not shown) for projecting a display onto a surface, such as, for example, a wearer's skin. In particular, such a display may be projected onto a wearer's forearm. As will be explained in greater detail below, such an embodiment may be useful for displaying or presenting an alert to a wearer. In some embodiments of the invention, a display such as that described may include functionality such as communication between device 100 and the wearer. In still other embodiments of the invention, such a display may facilitate communication as may be made using a mobile telephone, etc., in the case that device 100 further includes such functionality.

While device 100 is shown in FIGS. 1 and 2 as a bracelet-like device to be worn about a wearer's wrist, this is neither necessary nor essential. Other devices may be employed to monitor an individual's pulse. For example, such devices may include clothing items (e.g., shirts, socks, undergarments, hats, headbands, watches, rings, etc.) or other wearable devices (e.g., chest straps, etc.) with one or more pulse sensors incorporated therein. In still other embodiments of the invention, the functionality of such devices may be incorporated into an implantable device, such as a pacemaker, defibrillator, or similar device. In such an embodiment, the implantable device may be operable to indicate whether the pulse monitoring device has been activated. For purposes of simplicity and illustration only, device 100 will be shown and described as a bracelet-like device to be worn about a wearer's wrist.

In any event, HR sensors 120, 122 are operable to detect and/or measure a pulse of a wearer and communicate such detection and/or measurement to an electronic device, such as a mobile telephone. As one skilled in the art will recognize, a detected and/or measured pulse may be employed to determine either or both of a corresponding heart rate or a heart beat regularity.

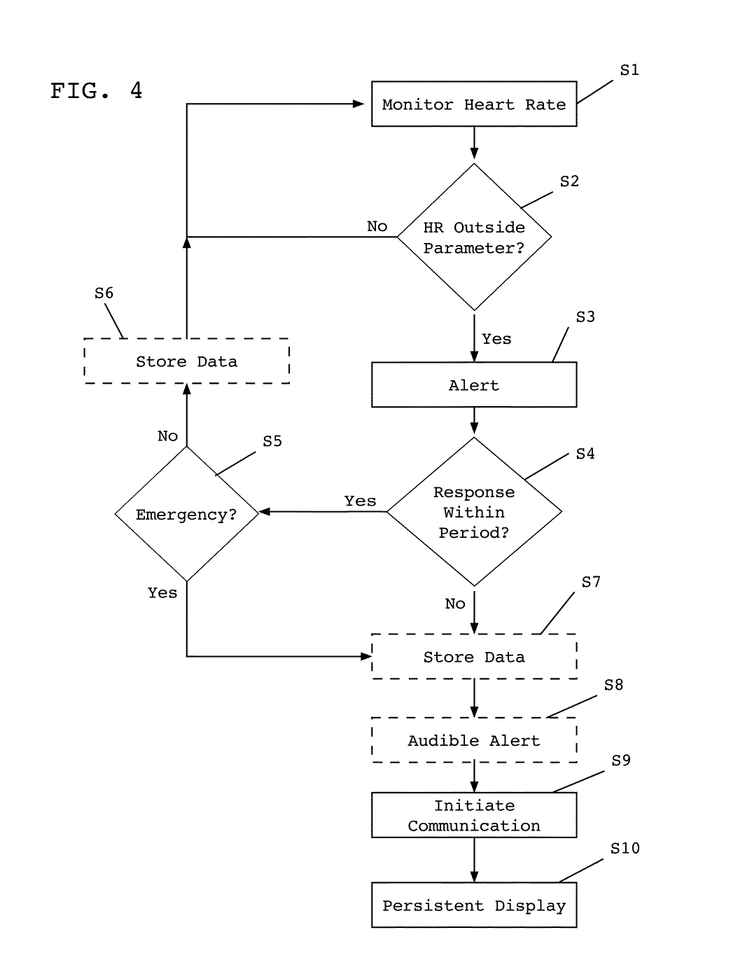

FIG. 3 shows a schematic view of device 100 and a mobile telephone 200, with communication 180 therebetween. As noted above, such communication 180 may be by way of, for example, Bluetooth communication. Other methods, mechanisms, means, or protocols for communication between device 100 and mobile telephone 200 may be employed, as will be recognized by one skilled in the art. Similarly, mobile telephone 200 is but one example of an electronic device with which device 100 may communicate. Other electronic devices include, for example, a tablet computer, a laptop computer, a desktop computer, a wearable electronic device, etc. For purposes of simplicity and illustration only, the communications device with which device 100 communicates will be described hereafter as a mobile telephone 200.

As shown in FIG. 3, mobile telephone 200 is configured for use with a mobile or satellite communications system 300. Mobile telephone 200 may communicate 280 with communications system 300 via, for example, a cellular or satellite system, a Voice over Internet Protocol (VoIP) system, a short message system (SMS) component of a cellular phone network, etc. In still other embodiments of the invention, device 100 and/or mobile telephone 200 may be configured for communication 182, 282, respectively, with or via a wireless Internet network 400. Wireless Internet network 400 may include or have connected to it a storage device 410 for storing and/or retrieving data collected by or from device 100 and/or mobile telephone 200. According to some embodiments, storage device 410 may include a cloud storage device (e.g., a remotely-located storage device to which data may be stored and/or retrieved).

As will be explained in greater detail below, upon detection of a pulse of a wearer that is outside a normal or predetermined parameter, device 100 may communicate 180 data regarding such pulse to mobile telephone 200 and/or communicate 182 such data to or via wireless Internet network 400. In the event that such data are communicated to mobile telephone 200, mobile telephone 200 may, according to some embodiments of the invention, initiate a communication 380,382 via communications system 300 with an emergency services provider 310 and/or one or more predetermined emergency contacts 320, respectively.

FIG. 4 shows a flow diagram of a method according to one embodiment of the invention. At S1, a wearer's pulse is monitored using a device 100 according to the invention. At S2 it is determined whether the pulse monitored at S1 is outside a parameter. Such a parameter may include, for example, a minimum heart rate (below which would indicate a low heart rate), a maximum heart rate (above which would indicate a high heart rate), a heart beat regularity (outside which would indicate an irregular heart beat), or the absence of a pulse. Such a parameter may be predetermined or may be calibrated to a particular individual during a “learning” period, which will be described in greater detail below. Regardless, if a monitored pulse is determined not to be outside a parameter (i.e., No at S2), flow reverts to S1 and pulse monitoring continues.

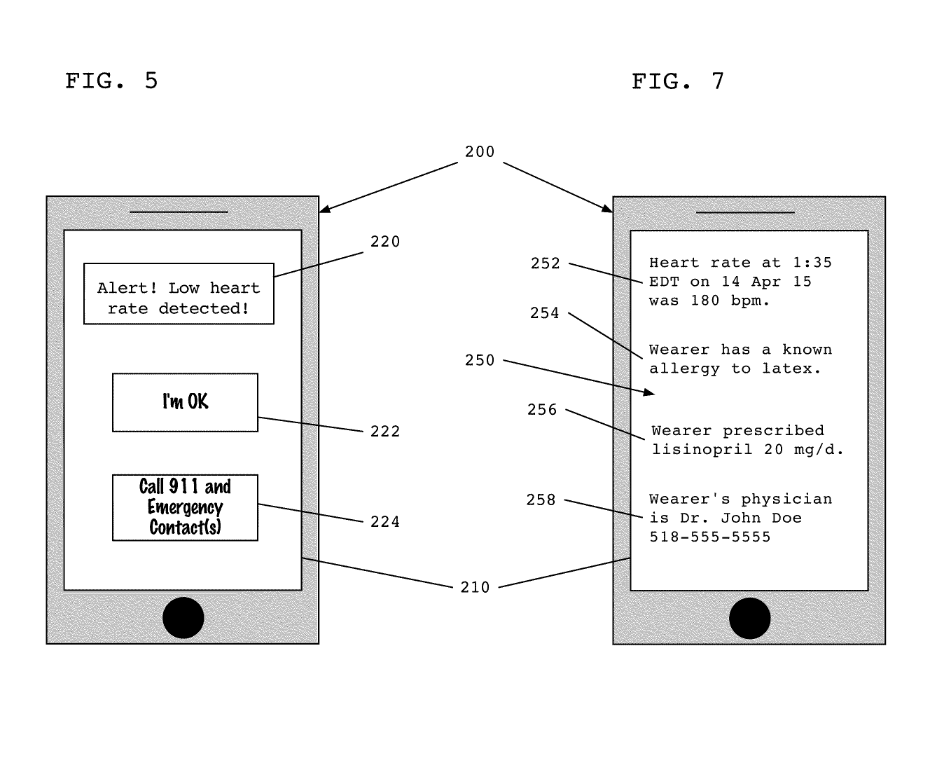

If, instead, a monitored pulse is determined at S2 to be outside a parameter (i.e., Yes at S2), the individual whose pulse is being monitored is alerted at S3. Such an alert may be made using, for example, the alert mechanism 132 (FIG. 1) of device 100 and/or mobile telephone 200 (FIG. 3) and may include, for example, a visual, a tactile (e.g., vibratory) signal and/or an audible signal capable of alerting the individual and those nearby the individual. For example, FIG. 5 shows a detailed view of mobile telephone 200, in which the screen 210 includes an alert 220 according to one embodiment of the invention. Here, screen 210 includes a visual alert 220 regarding the monitored pulse and further includes a first button 222 for an individual to indicate that they are not experiencing a medical emergency and a second button 224 for the individual to indicate that they are experiencing a medical emergency.

Returning to FIG. 4, it is determined whether a response from the individual is received within a predetermined period. Such a period may be preset or configured according to the individual's preferences. Such a period may be, for example, 10 seconds, 15 seconds, 30 seconds, 60 seconds, or the like. According to some embodiments of the invention, different periods for response may be set depending on the parameter that the monitored pulse is determined to be outside at S2.

For example, a high heart rate may be the result of the individual exercising or have some other cause that is not necessarily indicative of a medical emergency. In such case, the period for response by the individual may be longer than, for example, if the monitored pulse is determined to be irregular or if a pulse is not detected. Such instances may be more indicative of a medical emergency, in which case the period for response may be shorter. One skilled in the art will recognize that heart rates can vary greatly between and among individuals and that the applicable period(s) for response may similarly vary between and among individuals.

If a response is received from the individual within the requisite period (i.e., Yes at S4), it is determined at S5 whether the individual's response indicates that they are experiencing a medical emergency. If not (i.e., No at S5), data regarding the monitored pulse may optionally be stored at S6 and flow returns to S1. Data stored at S6 may be stored, for example, on device 100 itself, on mobile telephone 200, or on a storage device 410 (FIG. 3). According to some embodiments of the invention, storing data at, for example, S6, may include tagging or otherwise identifying such data for use in establishing or revising a pulse parameter, as will be explained in greater detail below.

If, on the other hand, it is determined that the individual's response indicates that they are experiencing a medical emergency (i.e., Yes at S5), data regarding the monitored pulse may again optionally be stored at S7. In addition, an audible alert may optionally be activated at S8 to alert bystanders, etc. to the wearer's situation. Similarly, if it is determined at S4 that the individual did not respond within the requisite period (i.e., No at S4), flow passes to S7 and S8 as noted above.

In either case, and whether or not data are stored at S7 or an alert provided at S8, flow then passes to S9, where a communication is initiated. As noted above, such communication may include a communication with an emergency services provider 310 (FIG. 3) and/or one or more emergency contacts 320 (FIG. 3) such as a spouse, other family member, or physician. Similarly, the communication itself may take any number of forms, including, for example, a telephone call, an SMS message, etc. According to some embodiments of the invention, a GPS location, determined using device 100 and/or mobile telephone 200 may be included in such a communication. According to other embodiments of the invention, data regarding the monitored pulse may also be provided as part of such a communication.

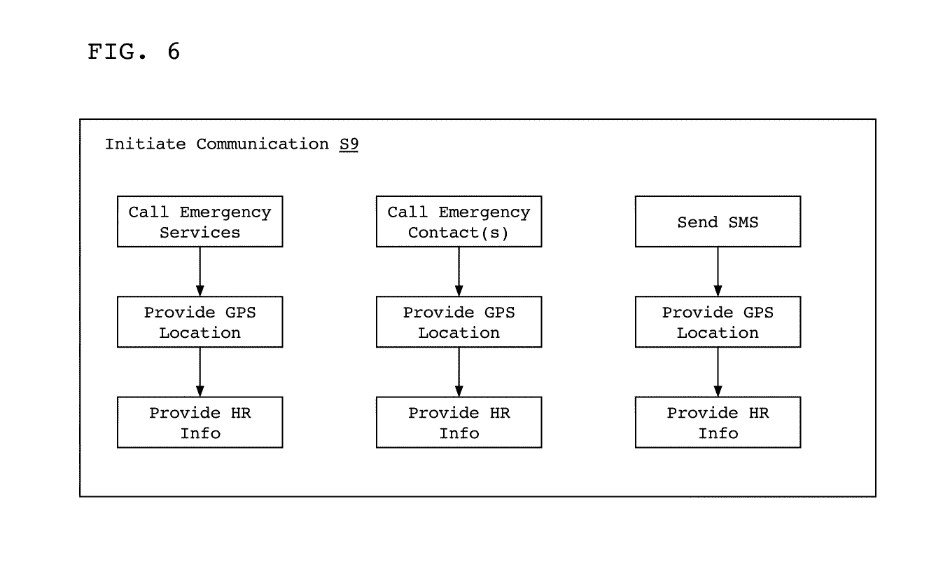

FIG. 6 shows an expanded view of S9 of FIG. 4. As can be seen in FIG. 6, communications initiated at S9 may include calling an emergency services provider, providing the emergency services provider with a GPS location of the individual, and providing data regarding the monitored pulse. Similar steps may be taken when contacting emergency contact(s) or when sending an SMS message.

In the case that a voice call to an emergency services provider and/or emergency contact is made as part of the communication initiated at S9, GPS location data and pulse data may be provided in any number of ways. For example, such data may be provided electronically or converted to an audible signal as part of the voice call. In the case that an SMS message is sent as part of the communication initiated at S9, such data are provided electronically and may optionally be converted to speech according to any number of methods known in the art.

Returning to FIG. 4, at S10, a persistent display may be forced on mobile telephone 200 in response to a determination that the individual either did not respond within the requisite period at S4 or responded that the individual was experiencing a medical emergency at S5. A persistent display on mobile telephone is not subject to a sleep period or other time out period that may be set on the mobile telephone and will continue to be displayed until acted upon by a user.

It should be noted that although S7-S10 are shown are shown in FIG. 4 as occurring in a particular order, this is neither necessary nor essential. For example, these steps may be carried out in a different order than that shown in FIG. 4 or may be carried out substantially simultaneously, as will be recognized by one skilled in the art.

FIG. 7 shows an example of such a persistent display on mobile telephone 200. Here, display 210 includes pulse data 252, allergy information regarding the individual 254, medications currently prescribed to the individual 256, and contact information for the individual's physician 258, any of which may be of critical importance to emergency services personnel responding to the communication initiated at S7.

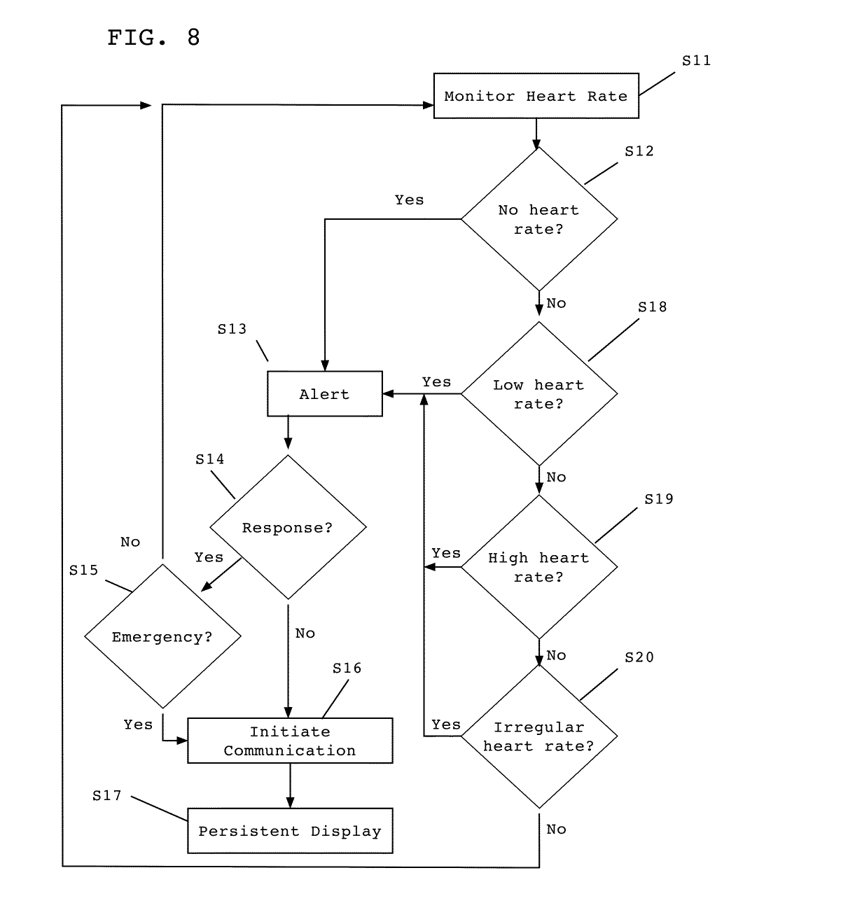

FIG. 8 shows a flow diagram of a method according to another embodiment of the invention. Here, an individual's pulse is monitored at S11, as described above. At S12, it is determined whether no pulse (asystole) is detected. If so (i.e., Yes at S12), the individual and others around the individual may be alerted at S13 as described above. It is determined at S14 whether a response from the individual was received within a requisite period, also as described above. If so (i.e., Yes at S14), it is determined at S15 whether the response indicates that the individual is experiencing a medical emergency. If not (i.e., No at S15), flow returns to S11. If the response does indicate that the individual is experiencing a medical emergency (i.e., Yes at S15), a communication is initiated at S16, as described above, and a persistent display may optionally be forced on the mobile telephone or other electronic device at S17, also as described above. In any case, data regarding the monitored pulse may optionally be stored at any point in the flow of FIG. 8, as described above, and as should be apparent from the description provided herein.

If, on the other hand, a pulse is detected (i.e., No at S12), it may then be determined at S18 whether the monitored pulse is consistent with being a low heart rate. If so (i.e., Yes at S18), flow passes to S13 as described above. If the monitored pulse is not consistent with being a low heart rate (i.e., No at S18), it may then be determined at S19 whether the monitored pulse is consistent with a high heart rate. If so (i.e., Yes at S19), flow again passes to S13 as described above. If the monitored pulse is not consistent with a high heart rate (i.e., No at S19), it may then be determined at S20 whether the monitored pulse is consistent with an irregular heart beat. If so (i.e., Yes at S20), flow again passes to S13 as described above. If the monitored pulse is not consistent with an irregular heart beat (i.e., No at S20), flow returns to S11.

One skilled in the art will recognize that the method shown in the flow diagram of FIG. 8 is subject to any number of modifications, all of which are within the scope of the invention. For example, the position of each of S12, S18, S19, and S20 may be exchanged with any other of this group. In addition, the step of storing data related to the monitored pulse, as noted above and shown at S6 of FIG. 4, may optionally be incorporated at any position within the flow diagram of FIG. 8. Similarly, upon indication that a communication should be initiated, an audible alert, as shown, for example, at S8 of FIG. 4, may also be incorporated into the flow of FIG. 8. One skilled in the art will recognize any number of other additions or modifications to the method described, all of which are intended to be within the scope of the invention.

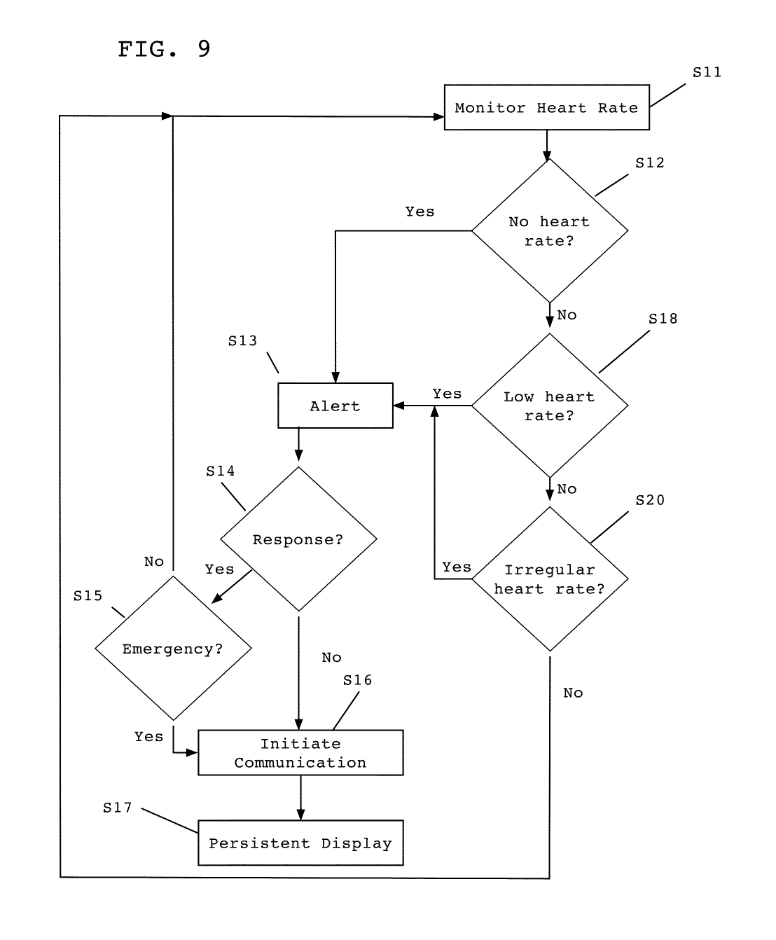

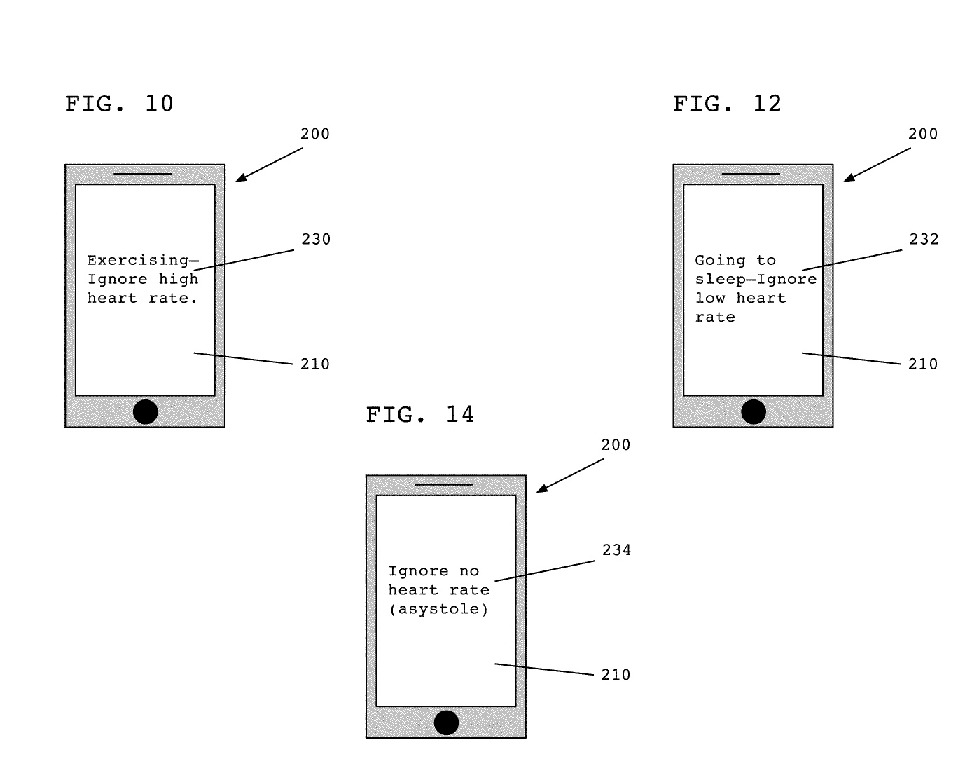

Similarly, various components of the method shown in FIG. 8 may be omitted for one reason or another. For example, an individual may desire to continue wearing device 100 while exercising. It is quite likely, however, that such exercise will increase the individual's heart rate to the extent that a high heart rate alert would be detected. FIG. 9 shows a flow diagram according to another embodiment of the invention in which the high heart rate determination has been omitted. FIG. 10 shows display 210 of mobile telephone 200 with a high heart rate bypass 230 in place.

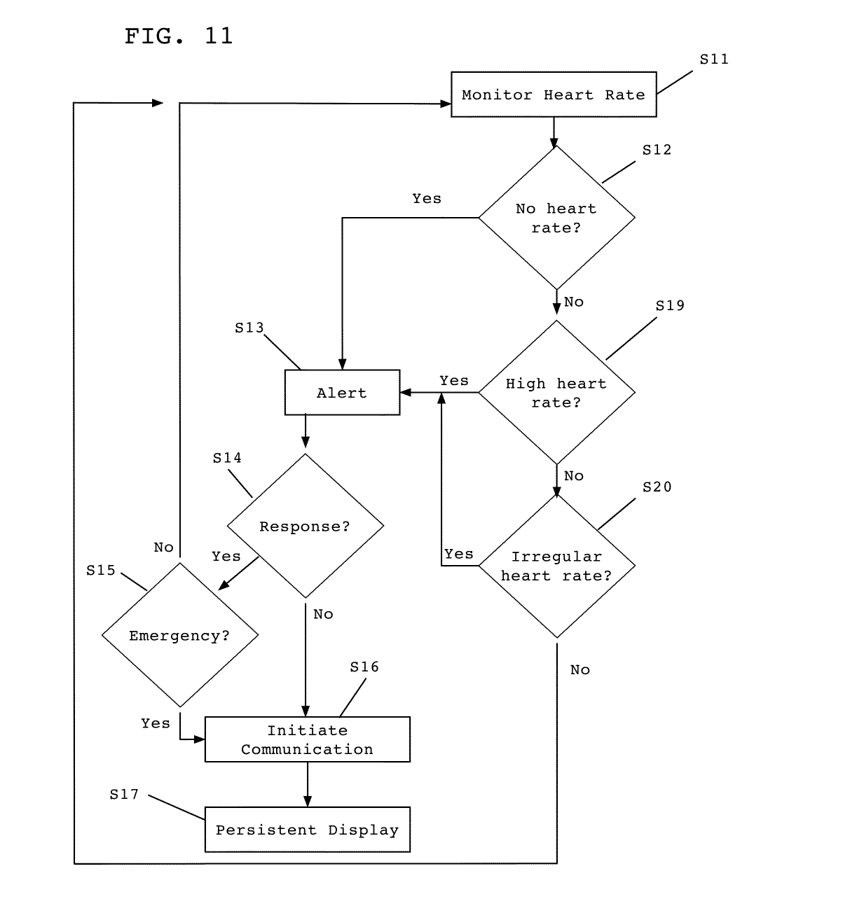

Other components of the method shown in FIG. 8 may similarly be omitted. For example, an individual may desire to continue wearing device 100 while sleeping. It is possible, however, that the individual's heart rate, during sleep, will decrease to the extent that a low heart rate alert would be detected. FIG. 11 shows a flow diagram according to another embodiment of the invention in which the low heart rate determination has been omitted. FIG. 12 shows display 210 of mobile telephone 200 with a low heart rate bypass 232 in place.

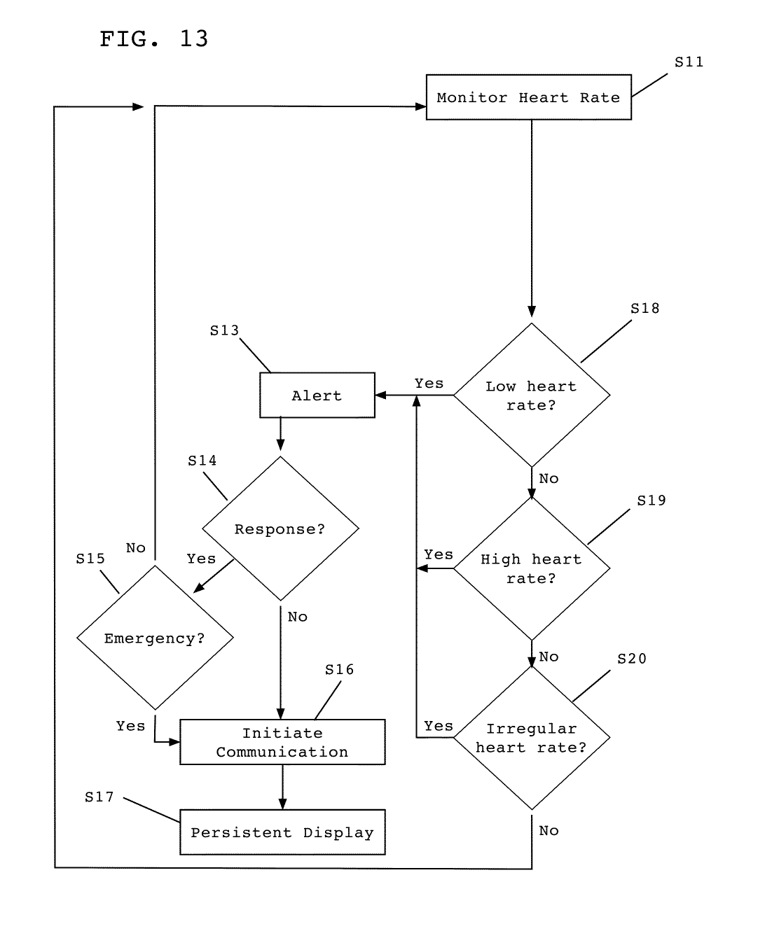

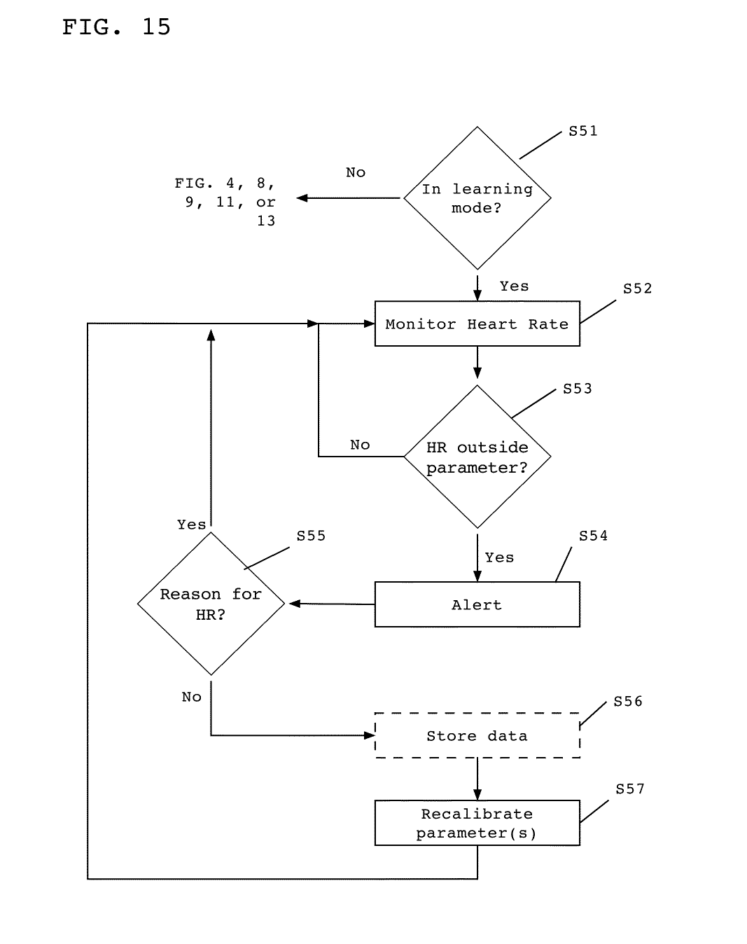

In other cases, an individual may desire not to be notified if no pulse is detected, but to be notified if a high, low, or irregular heart beat is detected. This may be the case, for example, where the individual intends to repeatedly remove device 100. FIG. 13 shows a flow diagram according to another embodiment of the invention in which the no pulse (asystole) determination has been omitted. FIG. 14 shows a display 210 of a mobile telephone 200 with a no pulse (asystole) bypass 234 in place.

With respect to the methods shown in FIGS. 9,11, and 13, it should be noted that the step of storing data related to the monitored pulse, as described above and shown at S6 of FIG. 4, may optionally be incorporated at any position within the flow diagram of any of FIG. 9,11, or 13. Similarly, upon indication that a communication should be initiated, an audible alert, as shown, for example, at S8 of FIG. 4, may also be incorporated into the flow of any of FIG. 9,11, or 13.

As noted above, the various parameters described herein (no pulse, high heart rate, low heart rate, irregular heart beat) may be preset using known average parameters or may be established with respect to each individual. To this extent, one embodiment of the invention includes a “learning mode” in which an individual's pulse is monitored and, optionally, alerts provided to the individual, but no communications are initiated.

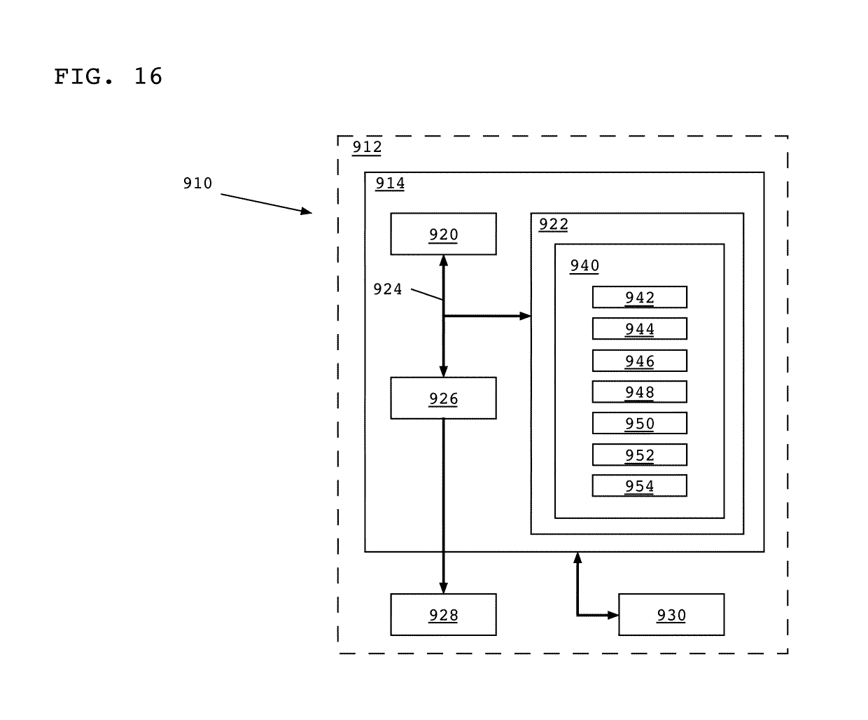

For example, FIG. 15 shows a flow diagram of such a method. Here, it is determined at S51 whether device 100 and/or mobile telephone 200 are in a learning mode. If not (i.e., No at S51), flow passes to one of, for example, FIG. 8,9,11, or 13. If the device and/or mobile telephone 200 are in learning mode (i.e. Yes at S51), the individual's pulse is monitored at S52, as described above. At S53, it is determined whether the monitored pulse is outside one or more parameter (e.g., no pulse, high heart rate, low heart rate, irregular heart beat), where such parameter is preset or based on default or average parameters. If not (i.e., No at S53), flow returns to S52.

If the monitored pulse is determined to be outside one or more parameter (i.e., Yes at S53), the individual can optionally be alerted to this at S54. The individual can then be prompted at S55 to indicate whether a reason exists for the pulse to be outside a parameter (e.g., exercising, sleeping, device not worn, etc.). If a reason exists (i.e., Yes at S55), flow may return to S52. The monitored pulse data would not want to be employed in calibrating the parameters to the individual, since a reason exists for the monitored pulse to be outside at least one parameter.

If a reason does not exist (i.e., No at S55), the monitored pulse data may optionally be stored at S56. At S57, the monitored pulse data are used to recalibrate the one or more parameter that the monitored pulse was determined at S53 to be outside. Flow may then return to S52 and the recalibrated parameter employed at S53. The steps shown in FIG. 15 may then be iterated any number of times in order to calibrate the parameters to the individual. According to some embodiments of the invention, such steps may be iterated until the monitored pulse is determined not to be outside one or more parameter (i.e., No at S53) for some predetermined period of time (e.g., 60 minutes, four hours, one day, one week, etc.).

FIG. 16 shows an illustrative system 910 for monitoring a pulse. To this extent, system 910 includes a computer infrastructure 912 that can perform the various process steps described herein for monitoring a pulse. In particular, computer infrastructure 912 is shown including a computer system 914 that comprises pulse monitoring system 940, which enables computer system 914 to monitor a pulse by performing the process steps of the invention.

Computer system 914 is shown including a processing unit 920, a memory 922, input/output (I/O) interfaces 926, and a bus 924. Further, computer system 914 is shown in communication with external devices 928 and a storage system 930. As is known in the art, in general, processing unit 920 executes computer program code, such as pulse monitoring system 940, that is stored in memory 922 and/or storage system 930. While executing computer program code, processing unit 920 can read and/or write data from/to memory 922, storage system 930, and/or I/O interface 926. Bus 924 provides a communication link between each of the components in computer system 914. External devices 928 can comprise any device that enables a user (not shown) to interact with computer system 914 or any device that enables computer system 914 to communicate with one or more other computer systems.

In any event, computer system 914 can comprise any general purpose computing article of manufacture capable of executing computer program code installed by a user (e.g., a personal computer, server, mobile telephone, tablet computer, handheld device, etc.). However, it is understood that computer system 914 and pulse monitoring system 940 are only representative of various possible computer systems that may perform the various process steps of the invention. To this extent, in other embodiments, computer system 914 can comprise any specific purpose computing article of manufacture comprising hardware and/or computer program code for performing specific functions, any computing article of manufacture that comprises a combination of specific purpose and general purpose hardware/software, or the like. In each case, the program code and hardware can be created using standard programming and engineering techniques, respectively.

Similarly, computer infrastructure 912 is only illustrative of various types of computer infrastructures for implementing the invention. For example, in one embodiment, computer infrastructure 912 comprises two or more computer systems (e.g., a server cluster) that communicate over any type of wired and/or wireless communications link, such as a network, a shared memory, or the like, to perform the various process steps of the invention. When the communications link comprises a network, the network can comprise any combination of one or more types of networks (e.g., the Internet, a wide area network, a local area network, a virtual private network, etc.). Regardless, communications between the computer systems may utilize any combination of various types of transmission techniques.

As previously mentioned, the pulse monitoring system 940 enables the computer system 914 to monitor a pulse. To this extent, the pulse monitoring system 940 is shown including a pulse detection system 942, pulse analysis system 944, pulse alert system 946, response system 948, communication system 950, and persistent display system 952. Operation of each of these systems is discussed above. The pulse monitoring system 940 may further include other system components 954 to provide additional or improved functionality to the pulse monitoring system 940. It is understood that some of the various systems shown in FIG. 16 can be implemented independently, combined, and/or stored in memory for one or more separate computer systems 914 that communicate over a network. Further, it is understood that some of the systems and/or functionality may not be implemented, or additional systems and/or functionality may be included as part of system 910.

While shown and described herein as a method, system, and device for monitoring a pulse, it is understood that the invention further provides various alternative embodiments. For example, in one embodiment, the invention provides a computer-readable medium that includes computer program code to enable a computer infrastructure to monitor a pulse. To this extent, the computer-readable medium includes program code, such as pulse monitoring system 940, that implements each of the various process steps of the invention. It is understood that the term “computer-readable medium” comprises one or more of any type of physical embodiment of the program code. In particular, the computer-readable medium can comprise program code embodied on one or more portable storage articles of manufacture (e.g., a compact disc, a magnetic disk, a tape, etc.), on one or more data storage portions of a computer system, such as memory 922 and/or storage system 930 (e.g., a fixed disk, a read-only memory, a random access memory, a cache memory, etc.), and/or as a transitory data signal traveling over a network (e.g., during a wired/wireless electronic distribution of the program code).

In another embodiment, the invention provides a business method that performs the process steps of the invention on a subscription, advertising, and/or fee basis. That is, a service provider could offer to monitor a pulse, as described above. In this case, the service provider can create, maintain, support, etc., a computer infrastructure, such as computer infrastructure 912, that performs the process steps of the invention for one or more customers. In return, the service provider can receive payment from the customer(s) under a subscription and/or fee agreement and/or the service provider can receive payment from the sale of advertising space to one or more third parties.

In still another embodiment, the invention provides a method of generating a system for monitoring a pulse. In this case, a computer infrastructure, such as computer infrastructure 912, can be obtained (e.g., created, maintained, having made available to, etc.) and one or more systems for performing the process steps of the invention can be obtained (e.g., created, purchased, used, modified, etc.) and deployed to the computer infrastructure. To this extent, the deployment of each system can comprise one or more of (1) installing program code on a computer system, such as computer system 914, from a computer-readable medium; (2) adding one or more computer systems to the computer infrastructure; and (3) incorporating and/or modifying one or more existing systems of the computer infrastructure, to enable the computer infrastructure to perform the process steps of the invention.

As used herein, it is understood that the terms “program code” and “computer program code” are synonymous and mean any expression, in any language, code or notation, of a set of instructions intended to cause a computer system having an information processing capability to perform a particular function either directly or after either or both of the following: (a) conversion to another language, code or notation; and (b) reproduction in a different material form. To this extent, program code can be embodied as one or more types of program products, such as an application/software program, component software/a library of functions, an operating system, a basic I/O system/driver for a particular computing and/or I/O device, and the like.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A pulse monitoring system comprising:
a monitoring device for monitoring a pulse of an individual, the device being operable to detect a pulse and transmit data related to the detected pulse;
a communication device operable to receive data from the monitoring device and initiate a communication; and
a program product which, when executed, is operable to carry out a method comprising:
receiving data related to the detected pulse;
analyzing the data related to the detected pulse and determining either or both of a heart rate or a heart beat regularity;
determining whether the data are indicative of a heart rate or heart beat outside a predetermined parameter; and
in the case that the data are indicative of a heart rate or heart beat being outside a predetermined parameter, initiating at least one of the following events: an alert to the individual, a communication, a transmission of data, or a storing of data.

2. The pulse monitoring system of claim 1, wherein the program product is stored on the monitoring device.

3. The pulse monitoring system of claim 1, wherein the program product is stored on the communication device.

4. The pulse monitoring system of claim 1, wherein the predetermined parameter includes at least one parameter selected from a group consisting of: a maximum heart rate, a minimum heart rate, an irregular heart beat, and a period in which no heart beat is detected.

5. The pulse monitoring system of claim 1, wherein the method further comprises:
determining whether the individual has responded to the alert.

6. The pulse monitoring system of claim 5, wherein determining whether the individual's response to the alert includes an indication that the individual is experiencing a medical emergency.

7. The pulse monitoring system of claim 5, wherein the method further comprises:
initiating at least one of an audible alert or a visible alert using at least one of the monitoring device or the communication device.

8. The pulse monitoring system of claim 1, wherein the method further comprises:
forcing a persistent display on the communication device in the case that the data are are indicative of a heart rate or a heart beat outside a predetermined parameter.

9. The pulse monitoring system of claim 1, wherein the method further comprises:
receiving an indication that at least one of a high heart rate or a low heart rate should not trigger the initiation of the alert to the individual or the communication.

10. The pulse monitoring system of claim 1, wherein the pulse monitoring device includes an implantable device operable to indicate whether the pulse monitoring device has been activated and at least one of the communication device or the program product is operable to determine whether the pulse monitoring device has been activated.

11. A method of monitoring a pulse of an individual, the method comprising:
receiving data related to a detected pulse of an individual;
analyzing the data related to the detected pulse and determining either or both of a heart rate or a heart beat regularity;
determining whether the data are indicative of a heart rate or heart beat being outside a predetermined parameter; and
in the case that the data is indicative of a heart rate or heart beat outside a predetermined parameter, initiating at least one of the following events: an alert to the individual, a communication, a transmission of data, or a storing of data.

12. The method of claim 11, wherein the predetermined parameter includes at least one parameter selected from a group consisting of: a maximum heart rate, a minimum heart rate, an irregular heart beat, and a period in which no heart beat is detected.

13. The method of claim 11, wherein the method further comprises:
determining whether the individual has responded to the alert.

14. The method of claim 13, wherein determining whether the individual's response to the alert includes an indication that the individual is experiencing a medical emergency.

15. The method of claim 11, wherein the method further comprises:
receiving an indication that at least one of a high heart rate or a low heart rate should not trigger the initiation of the alert to the individual or the communication.

16. A pulse monitoring device comprising:
a device for detecting a pulse of an individual; and
a transmission device for transmitting data related to the detected pulse.

17. The pulse monitoring device of claim 16, wherein the transmission device is capable of transmitting data to an external communication device.

18. The pulse monitoring device of claim 16, further comprising:
an alert device for alerting the individual if a detected pulse is outside a predetermined parameter.

19. The pulse monitoring device of claim 18, wherein the predetermined parameter includes at least one parameter selected from a group consisting of: a maximum heart rate, a minimum heart rate, an irregular heart beat, and a period in which no heart beat is detected.

20. A program product stored on a non-transitory computer-readable storage medium, which when executed, is operable to carry out a method, the method comprising:
receiving data related to a detected pulse of an individual;
analyzing data related to a detected pulse;
determining whether the data are indicative of a heart rate or a heart beat outside a predetermined parameter; and
initiating at least one of the following events: an alert to an individual, a communication, a transmission of data, or a storing of data.
